(19) United States
(12) Patent Application Publication
(10) Pub. No.: US 2026/0338587 A1
(43) Pub. Date: Oct. 29, 2026

(54) FLOW SENSOR TYPE DETERMINATION

(71) Applicant: Europlaz Technologies Limited, Southminster, Essex (GB)

(72) Inventors: Frede Jensen, Southminster, Essex (GB); Edmund O'Keeffe, Southminster, Essex (GB)

(73) Assignee: Europlaz Technologies Limited, Southminster, Essex (GB)

(21) Appl. No.: 19/491,079

(22) PCT Filed: Jun. 7, 2024
(86) PCT No.: PCT/EP2024/065826

§ 371 Date: Dec. 8, 2025

(30) Foreign Application Priority Data
Jun. 9, 2023 (GB) 2308650.7

(51) Int. Cl. G01F 1/696; A61B 5/087; G01F 1/69

(52) U.S. Cl. CPC G01F 1/696; A61B 5/0878; G01F 1/69; A61B 2562/08

(57) ABSTRACT

A respiratory gas flow rate sensor having a gas flow channel includes: a thermo-resistive sensing element located in the flow channel to detect rate of flow; an identification resistor electrically coupled to the sensing element, and an electrical interface to facilitate electrical connection of the flow sensor to a controller arranged to determine the identification resistor value and thereby identify the flow sensor type. A respiratory gas flow sensing system is also provided that includes: a gas flow sensor as noted above; and an electronic controller electrically connected to the gas flow sensor and arranged to determine the identification resistor value and thereby identify the flow sensor type.

### CROSS-REFERENCE TO RELATED APPLICATIONS

This application is the U.S. national stage application of International Application No. PCT/EP2024/065826, filed Jun. 7, 2024, which international application was published on Dec. 12, 2024, as WO 2024/252002 A 1 in the English language. The International Application claims priority to European Patent Application No. 2308650.7, filed Jun. 9, 2023. The international application and European application are all incorporated herein by reference, in their entirety.

### FIELD

The present invention relates to a respiratory gas flow sensor and to a respiratory gas flow sensor system for measuring gas flow rates to and from the lung of a patient during respiratory care. The present invention is concerned with the automatic determination of a flow sensor type to reduce the likelihood of operator error.

### BACKGROUND

Systems and methods for measuring the gas flow and gas composition in respiratory therapy are well known. An example of a bi-directional, constant temperature, anemometry flow sensor is described in U.S. Pat. No. 4,363,238 (William) and earlier in UK patent application GB1,512,290A (Agar). Such flow sensors enable high measurement sensitivity with relatively low flow resistance and have become a de facto industry standard in neonatal respiratory care. The sensor element of such sensors functions as part of a system with the other inter-dependent elements including an electronic circuit and microcontroller software, such as described in U.S. Pat. No. 5,069,066 (Djorup) and European application EP1,950,535A1 (Christensen).

For present purposes the phrase “thermo-resistive sensing element” refers to the thermo-resistive flow sensor member which effects the measurement function. “Tidal volume” is the volume of air inhaled and exhaled at each breath. “Deadspace” is a volumetric space in the flow sensor and associated tube connectors from which gas does not reach the patient's alveoli during inhalation and/or which buffers carbon dioxide rich gas from reaching the exhaust channel during exhalation.

Operation of the flow sensor relies upon the principle that the electrical response of the sensing element is exponentially proportional to gas velocity. Typically, signal conditioning and arithmetic and algorithmic processing is applied in converting the analogue gas velocity measurement into a digital representation of a directional volumetric flow rate. This conversion is a function of the cross-sectional area of the flow channel within the sensor body and gas density factors. The relationship between flow rate and time is used to derive the tidal breath volume (the volume of air inhaled and exhaled at each breath). The rate of respiratory flow change is further used to detect the onset of breath in spontaneous breathing patients and initiates ventilator synchronisation.

Different types of respiratory gas flow sensor are designed and marketed for different patient groups and combination devices. Neonatal patients, for example, require sensors in which the flow channel has a minimised deadspace to avoid rebreathing of exhaled gas causing harmful levels of blood carbon dioxide. Such a smaller flow channel presents a higher flow resistance, which can prevent full elastic recoil in the lungs of larger and adult patients, resulting in an incomplete exhalation between breaths. This causes an abnormal retention of air within the lungs, known as gas trapping. Sensors intended for neonatal, paediatric and adult patients are therefore designed with different flow channel dimensions.

Furthermore, flow sensors are often used in combination with sidestream capnometers or other gas sampling devices. A flow sensor may incorporate a sidestream port upstream from the flow sensing elements, such as described in patent U.S. Pat. No. 9,999,741 (Jensen); or a flow sensor may alternatively combine with a sidestream port adapter that is placed downstream from the flow sensing element. In the latter case, a ventilator usually incorporates an algorithm for compensating the respiratory tidal flow measurement for the lost sampling gas. In the case of ventilating small preterm neonates with, say, just 100 ml/minute volume, a typical sampling gas rate of 50 ml/min requires 50% compensation in calculating the tidal breath volume. The determination of the most appropriate sensor type for a specific patient is thus vital.

The last 20 years have seen an emerging trend in universal ventilators, with devices initially intended for paediatric and adult care being modified to enable small tidal volume ventilation of neonates; and with neonatal ventilators being modified to increase their therapeutic range to embrace larger paediatric size patients. In all cases, the operator of the ventilator or respiratory function monitor must manually select the flow sensor's patient type and/or type of combination device arrangement. An unmitigated selection error which results in a volume targeted ventilator applying an incorrect conversion and/or correction algorithm can potentially cause serious or catastrophic injury to a patient.

Commercial flow sensor models SFM3300 (neonatal, paediatric) and SFM3400 (adult) by Sensirion AG, Switzerland integrate the flow sensing elements and electronic controller (sometimes referred to as electronic driver) into the flow sensor body and present a common digital interface that makes the sensor types interchangeable, as far as the host device is concerned. This integration approach has some drawbacks, compared to a separation approach that segregates the flow sensing elements from the electronic controller. In the integration approach a single signal conversion function is digitally hardcoded into the specific sensor model. This does not in itself enable the host to detect if a large dead space adult flow sensor is being inappropriately used with a neonatal patient, including where the sensor is used in combination with a sidestream port adapter that is placed downstream from the flow sensing element. The integration of electronics into the disposable flow sensor body further results in printed circuit boards with electronic components entering the clinical waste stream, which is generally not recycled.

### SUMMARY

It is a principal aim of the present invention to provide a respiratory gas flow sensor and a respiratory gas flow sensor system which address at least some of the above-mentioned problems associated with human operator error and the determination of flow sensor types. The respiratory gas flow sensor is a respiratory gas flow rate sensor. This may be mass flow rate and/or volumetric flow rate.

According to a first aspect of the present invention there is provided a respiratory gas flow sensor having a flow channel formed therein, the sensor comprising:
a thermo-resistive sensing element located in the flow channel;
an identification resistor electrically coupled to the sensing element; and
an electrical interface to facilitate electrical connection to a controller arranged to determine the identification resistor value and thereby identify the flow sensor type.

The electrical interface is preferably a socket or an electronic cable connector providing connectivity to a host device. The host device may be a ventilator or a respiratory function monitor device.

The flow channel is a gas flow channel for the respiratory gas. The thermo-resistive sensing element senses flow rate of respiratory gases passing along the flow channel.

According to a closely related second arrangement of the present invention, there is provided a respiratory gas flow sensor system comprising:
a gas flow sensor as described herein having: a flow channel formed therein; a thermo-resistive sensing element located in the flow channel and an identification resistor electrically coupled to the sensing element; and
a controller electrically connected to the gas flow sensor and arranged to determine the identification resistor value and thereby identify the flow sensor type.

In broad aspects, the present invention provides advancements in safety and authentication of equipment for use on a patient in a medical context. The term patient as used herein refers to a human or an animal.

Preferably, the controller determines the flow sensor type automatically so that the operator does not need to manually select the flow sensor's patient type and/or type of combination device arrangement. This minimises the risk of unmitigated selection errors.

The sensor of the present invention is configured for connection to a controller which is remote therefrom. The controller may comprise an electronic driver/electronic circuit incorporating an identification resistor value detector. This arrangement enables the resistor value to be determined externally from the flow sensor. Since flow sensors are generally disposable, by providing the controller externally therefrom, the ingress of electronic components into the clinical waste stream is minimised. Furthermore, the provision of a controller distinct from the flow sensor allows a range of signal conversion functions to be pre-programmed externally from the connected sensor. In this way, the controller may be configured with the appropriate software to identify a range of different flow rate sensors.

The flow channel defines a flow path extending between a first end, configured to connect to a supply of gas, and a second end, configured to couple to a patient airway interface. The flow channel may ideally be a substantially straight passageway. This can help to minimise turbulence and improve flow characteristics. The flow channel may be tubular and usually is of a generally circular cross section, although it may have a different cross-sectional shape.

Preferably there are at least two thermo-resistive sensing elements and the identification resistor is electrically coupled to each. In this arrangement the identification resistor may be electrically coupled to either the current inflow or the current outflow ends of the two or more thermo-resistive sensing elements. Alternatively, the identification resistor is connected to the current inflow end of a first or first group of thermo-resistive sensing elements and the current outflow end of a second or second group of thermo-resistive sensing elements within said flow sensor.

The thermo-resistive sensing elements may comprise a wire, a flat bar or a film. The thermo-resistive sensing elements may be arranged perpendicularly across and/or substantially centrally within the flow path. Preferably the sensing elements are energised in accordance with principles for constant temperature anemometry or constant current anemometry or constant power anemometry.

Preferably the gas flow sensor system further comprises an electric cable electrically connecting the gas flow sensor to the controller. The cable may be up to 2 m in length.

In one arrangement, the controller of the gas flow sensor system may comprise an amplifier for determining the identification resistor value. The amplifier may be a high impedance differential amplifier. Preferably, the controller includes a Wheatstone Bridge to convert the resistance into a voltage differential and the high impedance differential amplifier amplifies that voltage difference.

In an alternative arrangement, the controller of the gas flow sensor system may comprise switches and may be configured to control the state of the switches to determine the identification resistor value.

Preferably, the resistive value of the identification resistor is greater than one Kilo Ohm. Depending upon the configuration of the flow sensor, this may prevent the identification resistor from interfering with the normal measurement operation of the gas flow sensor.

The identification resistor is ideally provided in the senor in a location distinct from the sensing element. The identification resistor is ideally not located in the gas flow path. The identification resistor may be in a part of the sensor external to the gas flow path. Among other advantages location outside the flow path ensures the gas flow is not disrupted. The gas flow sensor may define a port leading to the flow channel. That port may be a side port. Preferably, the identification resistor is housed within the port or within an insert located in the port so as to electrically connect to the sensing elements, yet preferably to be outside the gas flow path. The identification resistor may be provided in an aperture, and that aperture may be in the insert. The insert may also carry the sensing element(s). That could permit the sensing elements to project into the gas flow channel. The flow sensor may further comprise a cap, arranged to seal the resistor. The cap may comprise a compressible elastomeric cap. The seal provided by the cap may be configured to prevent the ingress of moisture to the resistor.

The respiratory gas flow sensor system of the present invention is preferably able to discriminate between more than fifty different sensor types and enables respiratory care ventilators and respiratory function monitors automatically to receive the correct tidal flow conversion and/or correction, without requiring any intervention from the human operator. It further enables host ventilators and monitors to detect sensors that are not clinically validated or otherwise unqualified for their specific application. This is an improvement over the prior art by preventing selection errors or incompatibility and thereby protecting the patient from potential sources of harm.

### BRIEF DESCRIPTION OF THE DRAWINGS

The present invention will now be described by way of example and with reference to the accompanying drawings in which:

FIG. 1 is a schematic view of a patient being ventilated by a ventilator set up including the flow sensor of the present invention and illustrates separation between the flow sensor and the controller which is remote therefrom;

FIG. 2 is a block schematic diagram of a first example electronic system that encompasses a flow sensor type identification;

FIG. 3 is a block schematic diagram of a second example electronic system that encompasses a flow sensor type identification;

FIG. 4 is a block schematic diagram illustrating possible alternative arrangements for coupling the identification resistor to a pair of thermo-resistive sensing elements;

FIG. 5 shows a detailed components and construction view of a flow sensor embodiment incorporating an identification resistor; and,

FIG. 6 illustrates a range of three flow sensor variants with differing operating characteristics while sharing identical cable connectors for connecting to a common electronic driver.

### DETAILED DESCRIPTION OF THE EMBODIMENTS

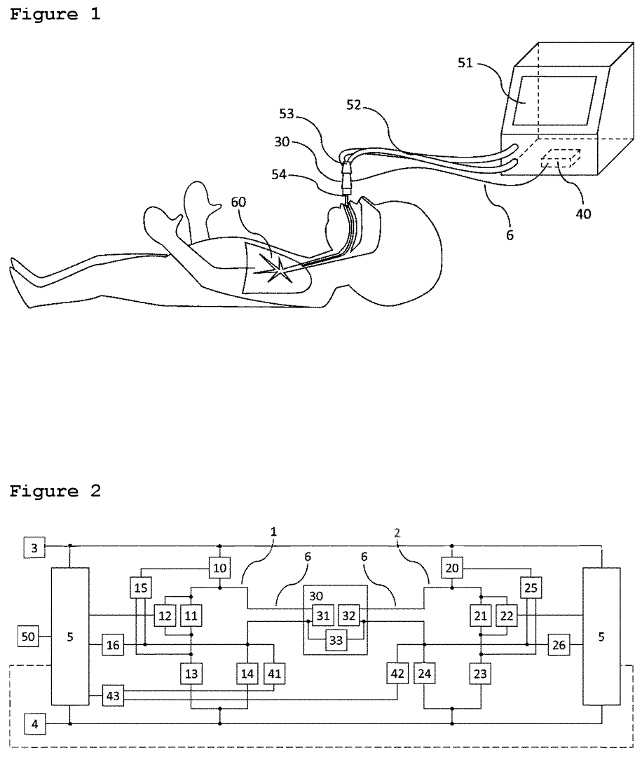

Referring initially to FIG. 1 there is shown a typical arrangement for clinical application where a ventilator 51 distal to the patient delivers respiratory gas through a ventilator breathing system 52. The ventilator breathing system connects to the flow sensor 30 via a manifold, often referred to as a Y-piece 53. The flow sensor is connected to an endotracheal tube 54 inserted into the patient's lung 60. The flow sensor 30 is connected via an electric cable 6 that is up to 2m in length to an electronic driver and detector unit 40 located inside (or associated with) the distal ventilator 51. The arrangement may have 2 operating phases. In the first phase, the flow sensor electronic driver 40 inside the ventilator 51 measures and identifies the flow sensor type. This process takes a few milliseconds to perform. In the second phase, normal flow measurement operation commences, where the electronic driver 40 inside the ventilator 51 applies the conversion algorithm and arithmetic that is specific to the identified flow sensor type.

In FIG. 2 there is shown an electronic system including an identification resistor 33 incorporated into a flow sensor 30. The system receives electrical power 3,4 and has a digital data bus interface 50 connecting to a host device, which may be a ventilator or respiratory function monitor. A microprocessor 5 shown in both the right-hand and left-hand side of FIG. 2 is a single combined component (explained by dotted line). As per conventional Constant Temperature Anemometry the thermo-resistive sensing element 31 and the three resistors 11, 13 and 14 form a Wheatstone bridge 1. A typical operating value of the thermo-resistive sensing element 31 and resistors 11, 13, 14 are in the regions of 4 Ohm, 8 Ohm, 3 kOhm and 5 kOhm respectively. The proportional integral amplifier 15 servo-controls transistor 10 to affect an electrical current that maintains the thermo-resistive sensing element 31 at an elevated constant temperature and resistance. A digitally programmable variable resistor 12 calibrates the bridge 1 to a target operating temperature. The mid-point value of the variable resistor 12 is typically equal to the value of resistor 11, to enable the calibration function straddling the 4/8 Ohm ratio of the low resistance bridge 1 leg. Gas flow across the thermo-resistive sensing element 31 causes a rise in the driving current, which is measurable as a voltage change in the output from signal conditioning amplifier 16. The analogue domain voltage of this signal represents the gas flow velocity and is digitised by the microcontroller's 5 analogue-to-digital converter. The system is mirrored with bridge 2 driving a second thermo-resistive sensing element 32. Variants of this conventional design may use additional heated or colder thermo-resistive elements, such as for gas temperature compensation. In the present invention the identification resistor 33 is inserted into the flow sensor 30 and is electrically connected between a pair of thermo-resistive sensing elements 31, 32. By temporarily setting the two digitally programmable variable resistors 12, 22 at different levels, such as zeroing the output from bridge 1 and setting bridge 2 to its lowest possible level, a known voltage differential appears across the identification resistor 33. The value of this resistor is represented by the analogue-to-digital signal that reaches the microcontroller 5 via a differential amplifier 43. To prevent the identification resistor 33 interfering with the normal measurement operation of the two servo-controlled bridges 1 and 2, its resistive value should ideally be more than 1 kOhm. Resistors 41, 42 and the differential amplifier 43 should for the same reason present a high impedance to the bridges 1 and 2.

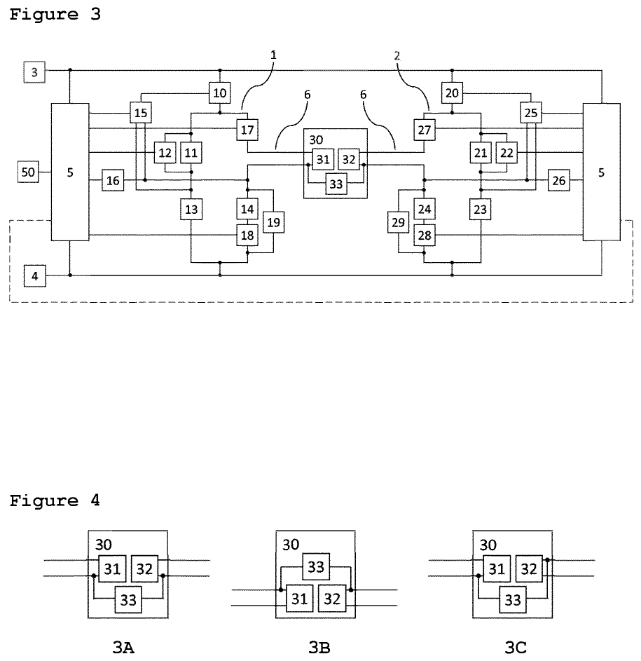

FIG. 3 shows an alternative example electronic system for measuring the value of identification resistor 33. Electronic components 17, 18, 27 and 28 are switches, such as milli-Ohm resistance MOSFET transistors or Solid State Relays or electro-mechanical Reed Relays. The four switches 17, 18, 27, 28 are kept in a low resistance state during normal flow measurement operation. When switch 17 is kept at low resistance while switches 18, 27 and 28 are switched to high resistance or open circuit, the voltage on the input to the signal conditioner 16 and the microprocessor's 5 analogue-to-digital converter is defined by the value of identification resistor 33 divided by the values of bypass resistors 19 and 29. The identity test can be reversed by setting switch 17 to high resistance and switch 18 to low resistance and then measuring the value from the mirrored signal conditioner 26. The electronic system in FIG. 3 enables a wider range of values for identification resistor 33, compared to the circuit in FIG. 2, and the test reversal offers a verification feature for improved assurance of correct identification. In an example embodiment of the system in FIG. 3, bypass resistors 19, 29 are each 10 kOhm. Such high resistive values significantly reduce the current flow through the Wheatstone bridges 1, 2 during the identification procedure. A logic high output from the microprocessor 5 to the proportional integral amplifier 15 assures transistor 10 fully conducts the low current. In this example embodiment, labelling the output from amplifier 16 as ADC1 and the output from amplifier 26 as ADC2, the identification resistor 33 value is found to be equal to 10000×(ADC2−ADC1)/ADC1. The identification resistor 33 may in this embodiment be specified in the range from 3 kOhm to 80 kOhm and reliably enable the system identifying more than fifty different sensor variants.

FIGS. 2 and 3 illustrates the servo-controller operating the principle of voltage feedback Constant Temperature Anemometry. Variant thermo-resistive sensing principles exists. Constant Current Anemometry is also well-know, for example, although it is less commonly used. Constant Current Anemometry maintains a target current through the thermo-resistive sensing elements 31,32 and permits varying temperatures. Constant Power Anemometry is mixed variant, in which a multiplier of voltage and current across the thermo-resistive sensing elements 31,32 is servo-controlled to be maintained constant. The flow sensor identification principle described for FIGS. 2 and 3 may equally be applied to such alternative variant thermo-resistive sensing principles. In all instances, the microcontroller 5 applies the type relevant conversion and correction to determine the volumetric flow rate and to output its digital value via a serial or parallel data bus interface 50 to a host device.

FIG. 4 illustrates alternative electrical coupling points of the identification resistor 33 to the thermo-resistive sensing elements 31 and 32 within the flow sensor 30. The identification method is practically insensitive to the alternative arrangements 3A, 3B and 3C and flexibly allows for the identification resistor 33 be connected to either the current inflow or the current outflow ends of the thermo-resistive sensing elements 31, 32.

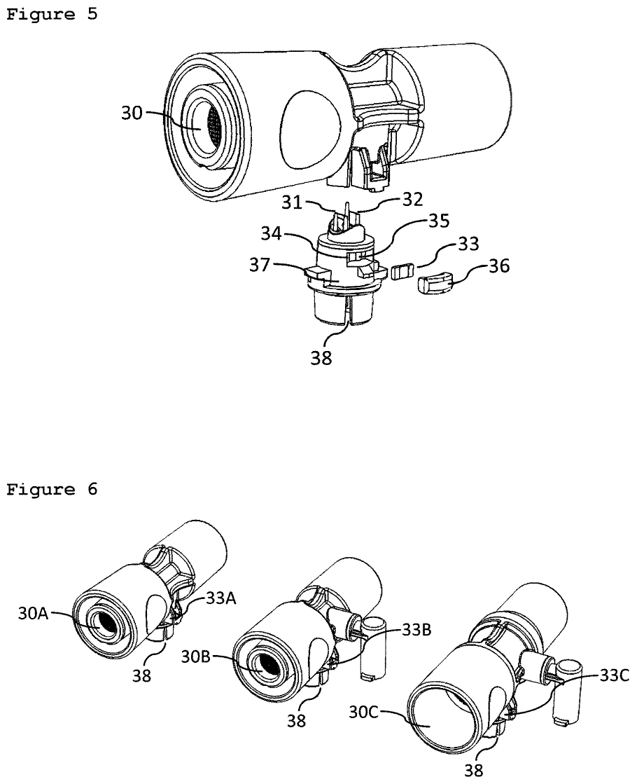

FIG. 5 illustrates an exploded view of an example embodiment of the identification resistor 33 about to be inserted into a typical respiratory flow sensor 30. The identification resistor 33 is a surface mount size 0805 resistor chip. The carrying insert 37 features a side aperture that extends into the exposed connector pins 34, 35. Once the insert 37 is guided and snapped into a port on the flow sensor body 30 a lightly compressed elastomeric cap (or bung) 36 pushes the identification resistor 33 into a forced interference contact with the connector pins 34, 35 which are in electrical contact with the thermo-resistive sensing elements 31, 32. The compression effect further deforms the elastomeric cap 36 to cause it to lightly spread in height and width and create a moisture seal against the insert's 37 cavity walls.

FIG. 6 illustrates a range of respiratory flow sensors 30A, 30B, 30C that all differ in dimensions or combination device port connectivity, while sharing the identical electronic cable connector 38 (as in FIG. 5) and system controller (electronic driver), as illustrated by FIGS. 2 and or 3. Flow sensor 30A may often be used in combination with a sidestream capnometry adapter (not shown) that draws a sampling gas downstream from the flow sensing element and therefore requires the tidal flow measurement be compensated for the lost respiratory gas. Flow sensor 30B incorporates a sidestream sampling port that is upstream from the flow sensing element, which eliminates the need for a correction factor and produces a net smaller dead space by also eliminating the additional sidestream adapter (not shown). Flow sensor 30C is a paediatric sensor enabling higher flow rates. Each flow sensor type is differentiated by each their own unique identification resistor value 33A, 33B, 33C.

1. A respiratory gas flow sensor having a flow channel formed therein, the sensor comprising:
a thermo-resistive sensing element located in the flow channel;
an identification resistor electrically coupled to the sensing element,
an electrical interface to facilitate electrical connection of the flow sensor to a controller arranged to determine the identification resistor value and thereby identify the flow sensor type,
wherein the identification resistor is housed within an aperture such that it is in electrical connection to the or each sensing element, and a cap is arranged to seal the aperture.

2. A respiratory gas flow sensor as claimed in claim 1, wherein there are at least two thermo-resistive sensing elements and the identification resistor is electrically coupled to each.

3. A respiratory gas flow sensor as claimed in claim 1 wherein the resistive value of the identification resistor is greater than one Kilo Ohm.

4. (canceled)

5. (canceled)

6. A respiratory gas flow sensor as claimed in claim 1 wherein the cap is a compressible elastomeric cap.

7. A respiratory gas flow sensor as claimed in claim 1, wherein the seal provided by the cap is configured to prevent the ingress of moisture into the aperture.

8. A respiratory gas flow sensor system comprising:
a gas flow sensor as claimed in claim 1 having: a flow channel formed therein; a thermo-resistive sensing element located in the flow channel and an identification resistor electrically coupled to the sensing element; and
an electronic controller electrically connected to the gas flow sensor and arranged to determine the identification resistor value and thereby identify the flow sensor type.

9. A respiratory gas flow sensor system as claimed in claim 6, wherein the gas flow sensor comprises at least two thermo-resistive sensing elements and the identification resistor is electrically coupled to each.

10. A respiratory gas flow sensor system as claimed in claim 6, further comprising an electric cable electrically connecting the gas flow sensor to the electronic controller.

11. A respiratory gas flow sensor system as claimed in claim 6, wherein the electronic controller comprises a high impedance differential amplifier for determining the identification resistor value.

12. A respiratory gas flow sensor system as claimed in claim 6, wherein the electronic controller comprises switches and is configured to control the state of the switches to determine the identification resistor value.

13. A respiratory gas flow sensor system as claimed in claim 6, wherein the resistive value of the identification resistor is greater than one Kilo Ohm.

14. (canceled)

15. (canceled)

16. A respiratory gas flow sensor system as claimed in claim 6 wherein the cap is a compressible elastomeric cap.

17. A respiratory gas flow sensor as claimed in claim 6, wherein the seal provided by the cap is configured to prevent the ingress of moisture into the aperture.

18. A respiratory gas flow sensor as claimed in claim 12, wherein the seal provided by the cap is configured to prevent the ingress of moisture into the aperture.
